(19) United States
(12) Patent Application Publication
(10) Pub. No.: US 2011/0189281 A1
(43) Pub. Date: Aug. 4, 2011

(54) TELMISARTAN AND HYDROCHLOROTHIAZIDE COMBINATION THERAPY

(75) Inventors: Helmut E. SCHUMACHER, Ingelheim (DE); Axel RIEDEL, Maselheim (DE); Peter BOEHM, Bad Kreuznach (DE)

(73) Assignee: BOEHRINGER INGELHEIM INTERNATIONAL GMBH, Ingelheim am Rhein (DE)

(21) Appl. No.: 13/087,887

(22) Filed: Apr. 15, 2011

Related U.S. Application Data
(63) Continuation of application No. 11/300,947, filed on Dec. 15, 2005, now abandoned.
(60) Provisional application No. 60/637,042, filed on Dec. 17, 2004.

(51) Int. Cl. A61K 31/5415; A61K 9/20; A61P 9/12; B65D 73/00

(52) U.S. Cl. 424/465; 514/223.5; 206/461

(57) ABSTRACT

A pharmaceutical composition comprising about 80 mg of telmisartan or a salt thereof and about 25 mg of hydrochlorothiazide or about 160 mg of telmisartan or a salt thereof and about 50 mg of hydrochlorothiazide, and methods of treating hypertension in patients with such combination.

### RELATED APPLICATIONS

This application claims benefit of U.S. Ser. No. 60/637,062, filed Dec. 17, 2004, which is hereby incorporated by reference in its entirety.

### FIELD OF THE INVENTION

The present invention relates to a pharmaceutical composition comprising as active ingredients about 80 mg of the angiotensin II receptor antagonist (ARB) telmisartan and about 25 mg of the diuretic hydrochlorothiazide (HCTZ) and a pharmaceutical composition which comprises about 160 mg telmisartan and about 50 mg hydrochlorothiazide and can be split into halves. The composition is used to treat hypertension in patients with an insufficient blood pressure reduction upon treatment either with an angiotensin II receptor antagonist, due to low plasma levels of renin or with a pharmaceutical composition of an angiotensin II receptor antagonist and lower doses of hydrochlorothiazide.

### BACKGROUND OF THE INVENTION

Telmisartan, a white to slightly yellowish solid, is an angiotensin II receptor antagonist developed for the treatment of hypertension and other medical indications as disclosed in EP-A-502314. It is a nonpeptide molecule chemically described as 4′-[(1,4′-dimethyl-2′-propyl-[2,6′-bi-1H-benzimidazol]-1′-yl)methyl]-[1,1′-biphenyl]-2-carboxylic acid or 4′-[2-n-propyl-4-methyl-6-(1-methylbenzimidazol-2-yl)benzimidazol-1-ylmethyl]biphenyl-2-carboxylic acid. Telmisartan's empirical formula is C₃₃H₃₀N₄O₂, its molecular weight is 514.63, and its structural formula is:

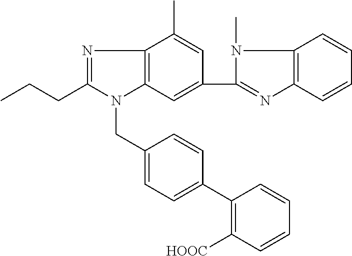

Telmisartan is manufactured and supplied in the free acid form. It is characterized by its very poor solubility in aqueous systems at the physiological pH range of the gastrointestinal tract of between pH 1 to 7. As disclosed in WO 00/43370, crystalline telmisartan exists in two polymorphic forms having different melting points. Under the influence of heat and humidity, the lower melting polymorph B transforms irreversibly into the higher melting polymorph A.

Hydrochlorothiazide (HCTZ), a white, odorless, crystalline powder with a molecular weight of 297.74, is a diuretic used in the treatment of edema and hypertension. HCTZ is chemically described as 6-chloro-3,4-dihydro-2H-1,2,4-benzothiadiazine-7-sulfonamide-1,1-dioxide. Its empirical formula is C₇H₈ClN₃O₄S₂, and its structural formula is:

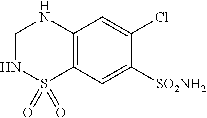

### OBJECT OF THE INVENTION

There exists a striking relationship between high blood pressure and cardiovascular morbidity and mortality, i.e., an increased risk of myocardial infarction, heart failure, stroke, or kidney disease in case of increased blood pressure. An incremental increase of 20 mmHg in systolic or 10 mmHg in diastolic blood pressure in individuals between 40 and 70 years of age doubles the risk of cardiovascular diseases. Therefore a target blood pressure of <140/90 mmHg is recommended and an even lower target of <130/80 mmHg for patients with co-morbidities such as diabetes or chronic kidney disease.

Many patients require two or more antihypertensive drugs to achieve this goal. One of the possible combination partners are thiazide-diuretics which are able to facilitate salt and water excretion. For a combination of an angiotensin II receptor antagonist (ARB) with HCTZ, a synergistic BP-lowering effect has been reported while additional ARB treatment results in almost no additional side effects. Available combination products combine the ARB with either a low dose of HCTZ or a high dose of HCTZ, wherein low dose means less then 15 mg, preferably 12.5 mg of HCTZ, and high dose means more than 15 mg, preferably 25 mg of HCTZ. Unfortunately a subgroup of hypertensive patients does still not adequately respond to treatment with an ARB or a combination therapy of an ARB plus a low dose diuretic, meaning that not in all patients the target blood pressure levels are achieved as suggested by the most recent guidelines, especially for patients with co-morbidities. In patients affected low plasma renin activity (PRA) is frequently observed. Renin is an enzyme released by the kidney to help control the body's sodium-potassium balance, fluid volume, and blood pressure. Renin itself is not actually measured in the PRA test, because it is difficult to measure renin in routine lab assay. In the most commonly used renin assay, the test actually determines, by a procedure called radioimmunoassay, the rate of angiotensin I generation per unit time, while the plasma renin concentration (PRC) measures the maximum renin effect. Both the PRA and the PRC are difficult to measure. Not only is renin itself unstable, but the patient's body position and the time of day affect the results. Also, the sample must be collected properly: drawn into a chilled syringe and collection tube, placed on ice, and sent to the performing laboratory immediately. Even if all these procedures are followed, results can vary significantly.

The current invention is based on the surprising finding, that administering a daily dose of 80 mg of telmisartan in combination with 25 mg instead of 12.5 mg of hydrochlorothiazide, results in an unexpected strong increase of the responder rate compared to patients treated with telmisartan or another ARB such as candesartan cilexetil, eprosartan, irbesartan, losartan, olmesartan, pratosartan, ripisartan, telmisartan, valsartan, or zolasartan or a combination of those, including telmisartan, with a low dose of the diuretic HCTZ. Therefore it is the object of the present invention to provide a further treatment option for patients whose blood pressure has not been adequately controlled with an ARB or a combination of an ARB with the low dose of the diuretic HCTZ. This option comprises the manufacture of a pharmaceutical composition comprising 80 mg telmisartan and 25 mg hydrochlorothiazide or a pharmaceutical composition which comprises about 160 mg telmisartan and about 50 mg hydrochlorothiazide and can be split into halves.

### DEFINITIONS

As used herein, the term “substantially amorphous” refers to a product comprising amorphous constituents in a proportion of at least 90%, preferably at least 95%, as determined by X-ray powder diffraction measurement.

The term “dissolving tablet matrix” refers to a pharmaceutical tablet base formulation having instant release (fast dissolution) characteristics that readily dissolves in a physiological aqueous medium.

The term “disintegrating tablet matrix” refers to a pharmaceutical tablet base formulation having instant release characteristics that readily disintegrates in a physiological aqueous medium.

### DETAILED DESCRIPTION OF THE INVENTION

The present invention comprises a pharmaceutical composition which can be used in a method for the treatment of hypertension comprising as active ingredients about 80 mg of the angiotensin II receptor antagonist telmisartan and about 25 mg of the diuretic hydrochlorothiazide and another pharmaceutical composition for the treatment of hypertension, which can be split into halves, comprising as active ingredients about 160 mg of telmisartan and about 50 mg hydrochlorothiazide.

The active ingredient telmisartan is generally supplied in its free acid form, although pharmaceutically acceptable salts such as the sodium salt may also be used. Since during subsequent processing telmisartan is normally dissolved and transformed into a substantially amorphous form, its initial crystal morphology and particle size are frequently of little importance for the physical and biopharmaceutical properties of the pharmaceutical formulation. It is, however, preferred to remove agglomerates from the starting material, e.g., by sieving, in order to facilitate wetting and dissolution during further processing.

The diuretic is usually used as a fine-crystalline powder, optionally in fine-milled, peg-milled or micronized form. For instance, the particle size distribution of hydrochlorothiazide, as determined by the method of laser light scattering in a dry dispersion system (Sympatec Helos/Rodos, focal length 100 mm) is preferably as follows:
d₁₀: ≦20 μm, preferably 2 to 10 μm
d₅₀: 5 to 50 μm, preferably 10 to 30 μm
d₉₀: 20 to 100 μm, preferably 40 to 80 μm

Preferred embodiments of the above pharmaceutical composition are tablets or capsules. Particularly preferred are bilayer tablets consisting of a first tablet layer comprising telmisartan in a dissolving tablet matrix having instant release (fast dissolution) characteristics and a separate second tablet layer comprising the active ingredient HCTZ in a disintegrating tablet matrix. The dissolving tablet matrix may have neutral or basic properties, although a basic tablet matrix is preferred.

Conveniently the composition of the present invention comprises telmisartan in substantially amorphous form which may be produced by any suitable method known to those skilled in the art, for instance, by freeze drying of aqueous solutions, coating of carrier particles in a fluidized bed, and solvent deposition on sugar pellets or other carriers. Preferably, however, the substantially amorphous telmisartan is prepared by the specific spray-drying method described in WO 03/059327.

Additionally, the composition of the present invention preferably comprises as inactive ingredients sodium hydroxide, meglumine, povidone, sorbitol, magnesium stearate, lactose monohydrate, microcrystalline cellulose, maize starch, and sodium starch glycolate.

A dissolving matrix of a telmisartan tablet layer may comprise a basic agent, a water-soluble diluent and, optionally, other excipients and adjuvants.

Specific examples of suitable basic agents are alkali metal hydroxides such as NaOH and KOH; basic amino acids such as arginine and lysine; and meglumine (N-methyl-D-glucamine), NaOH and meglumine being preferred.

Specific examples of suitable water-soluble diluents are carbohydrates such as monosaccharides like glucose; oligosaccharides like sucrose, anhydrous lactose, and lactose monohydrate; and sugar alcohols like sorbitol, mannitol, erythrol, and xylitol. Sorbitol is a preferred diluent.

The other excipients and/or adjuvants are, for instance, selected from binders, carriers, fillers, lubricants, flow control agents, crystallization retarders, solubilizers, coloring agents, pH control agents, surfactants and emulsifiers, specific examples of which are given below in connection with the second tablet layer composition. The excipients and/or adjuvants for a telmisartan tablet layer composition are preferably chosen such that a non-acidic, fast dissolving tablet matrix is obtained.

Such a first tablet layer composition generally comprises 3 to 50 wt. %, preferably 5 to 35 wt. %, of active ingredient; 0.25 to 20 wt. %, preferably 0.40 to 15 wt. %, of basic agent; and 30 to 95 wt. %, preferably 60 to 80 wt. % of water-soluble diluent (filler).

Other (optional) constituents may, for instance, be chosen from one or more of the following excipients and/or adjuvants in the amounts indicated:
10 to 30 wt. %, preferably 15 to 25 wt. %, of binders, carriers and fillers, thereby replacing the water-soluble diluent;
0.1 to 5 wt. %, preferably 0.5 to 3 wt. %, of lubricants;
0.1 to 5 wt. %, preferably 0.3 to 2 wt. %, of flow control agents;
1 to 10 wt. %, preferably 2 to 8 wt. %, of crystallization retarders;
1 to 10 wt. %, preferably 2 to 8 wt. %, of solubilizers;
0.05 to 1.5 wt. %, preferably 0.1 to 0.8 wt. %, of coloring agents;
0.5 to 10 wt. %, preferably 2 to 8 wt. %, of pH control agents; and
0.01 to 5 wt. %, preferably 0.05 to 1 wt. %, of surfactants and emulsifiers.

Such a telmisartan tablet layer may be produced by spray-drying an aqueous solution comprising telmisartan and a basic agent to obtain a spray-dried granulate, mixing the spray-dried granulate with a water-soluble diluent to obtain a premix, mixing the premix with a lubricant to obtain a final blend and compressing the final blend to form the first tablet layer.

A separate second tablet layer comprising HCTZ in a fast disintegrating tablet matrix preferably comprises one or more fillers, a binder or polymer, a disintegrant, a lubricant and, optionally, other excipients and adjuvants.

Preferred fillers are selected from the group consisting of pregelatinized starch, microcrystalline cellulose, low-substituted hydroxypropylcellulose, cellulose, mannitol, erythritol, lactose, saccharose, calcium hydrogen phosphate, sorbitol, and xylitol. Particularly preferred are pregelatinized starch, microcrystalline cellulose, mannitol, and lactose monohydrate. Particularly preferred are anhydrous lactose, spray-dried lactose, and lactose monohydrate.

Preferred disintegrants are selected from the group consisting of croscarmellose sodium salt (cellulose carboxymethyl ether sodium salt, crosslinked), sodium starch glycolate, crosslinked polyvinylpyrrolidone (crospovidone), corn starch, and low-substituted hydroxypropylcellulose. Particularly preferred are sodium starch glycolate and croscarmellose sodium salt.

Preferred binders are selected from the group consisting of polyvinyl pyrrolidone (Povidone), copolymers of vinylpyrrolidone with other vinyl derivatives (Copovidone), hydroxypropyl methylcellulose, methylcellulose, hydroxypropyl cellulose, and low-substituted hydroxypropyl cellulose. Particularly preferred are hydroxypropyl methylcellulose and Copovidone.

Preferred lubricants are sodium stearyl fumarate and magnesium stearate.

The other excipients and adjuvants, if used, are preferably selected from:
diluents and carriers such as cellulose powder, microcrystalline cellulose, cellulose derivatives like hydroxymethylcellulose, hydroxyethylcellulose, hydroxypropylcellulose and hydroxypropyl methylcellulose, dibasic calcium phosphate, corn starch, pregelatinized starch, polyvinyl pyrrolidone (Povidone) etc.;
lubricants such as stearic acid, magnesium stearate, sodium stearyl fumarate, glycerol tribehenate, etc.;
flow control agents such as colloidal silica, talc, etc.;
crystallization retarders such as Povidone, etc.;
solubilizers such as Pluronic, Povidone, etc.;
coloring agents, including dyes and pigments such as Iron Oxide Red or Yellow, titanium dioxide, talc, etc.;
pH control agents such as citric acid, tartaric acid, fumaric acid, sodium citrate, dibasic calcium phosphate, dibasic sodium phosphate, etc.;
surfactants and emulsifiers such as Pluronic, polyethylene glycols, sodium carboxymethyl cellulose, polyethoxylated, and hydrogenated castor oil, etc.; and
antioxidants,
and mixtures of two or more of these excipients and/or adjuvants.

The layers can be differentiated by using different colors.

A separate second tablet layer comprising HCTZ generally comprises 1.5 to 35 wt. %, preferably 2 to 15 wt. %, of active ingredient; 25 to 75 wt. %, preferably 35 to 65 wt. %, of filler; 10 to 40 wt. %, preferably 15 to 35 wt. %, of dry binder; 0.5 to 5 wt. %, preferably 1 to 4 wt. %, of wet granulation binder; and 1 to 10 wt. %, preferably 2 to 8 wt. %, of disintegrant. The other excipients and adjuvants are generally employed in the same amount as in the first tablet layer composition.

For preparing a bilayer tablet according to the present invention, the first and second tablet layer compositions may be compressed in the usual manner in a bilayer tablet press, e.g., a high-speed rotary press in a bilayer tabletting mode. However, care should be taken not to employ an excessive compression force for the first tablet layer. Preferably, the ratio of the compression force applied during compression of the first tablet layer to the compression force applied during compression of both the first and second tablet layers is in the range of from 1:10 to 1:2. For instance, the first tablet layer may be compressed at moderate force of 4 to 8 kN, whereas the main compression of first plus second layer is performed at a force of 10 to 20 kN.

During bilayer tablet compression adequate bond formation between the two layers is achieved by virtue of distance attraction forces (intermolecular forces) and mechanical interlocking between the particles.

The tablets obtained release the active ingredients rapidly and in a largely pH-independent fashion, with complete release occurring within less than 60 min and release of the major fraction occurring within less than 15 minutes. The dissolution/disintegration kinetics of the multilayer tablet may be controlled in different ways. For instance, the layers may dissolve/disintegrate simultaneously. Preferably, the tablet layer comprising HCTZ disintegrates first whereas the layer comprising telmisartan dissolves subsequently.

In accordance with the present invention, at least 70% and typically at least 90% of the active ingredients are dissolved after 30 minutes.

Bilayer tablets according to the present invention tend to be slightly hygroscopic and are therefore preferably packaged using a moisture-proof packaging material such as aluminum foil blister packs, or polypropylene tubes and HDPE bottles which preferably contain a desiccant.

A preferred method of producing the bilayer tablet according to the present invention comprises:
(i) providing a first tablet layer composition by:
   a) preparing an aqueous solution of telmisartan, at least one basic agent and, optionally, a solubilizer and/or a crystallization retarder;
   b) spray-drying the aqueous solution to obtain a spray-dried granulate;
   c) mixing the spray-dried granulate with a water-soluble diluent to obtain a premix;
   d) mixing the premix with a lubricant to obtain a final blend for the first layer; and
   e) optionally, adding other excipients and/or adjuvants in any of steps a) to d);
(ii) providing a second tablet comprising HCTZ;
(iii) compressing both the first and the second tablet layer composition to form a tablet layer; and
(iv) compressing the separate tablet layers to form a bilayer tablet.

To provide a first tablet layer composition, an aqueous alkaline solution of telmisartan is prepared by dissolving the active ingredient in purified water with the help of one or more basic agents like sodium hydroxide and meglumine Optionally, a solubilizer and/or a recrystallization retarder may be added. The dry matter content of the starting aqueous solution is generally 10 to 40 wt. %, preferably 20 to 30 wt. %.

The aqueous solution is then spray-dried at room temperature or preferably at increased temperatures of, for instance, between 50° C. and 100° C. in a co-current or countercurrent spray-drier at a spray pressure of, for instance, 1 to 4 bar. Generally speaking, the spray-drying conditions are preferably chosen in such a manner that a spray-dried granulate having a residual humidity of ≦5 wt. %, preferably ≦3.5 wt. %, is obtained in the separation cyclone. To that end, the outlet air temperature of the spray-drier is preferably kept at a value of between about 80° C. and 90° C. while the other process parameters such as spray pressure, spraying rate, inlet air temperature, etc. are adjusted accordingly.

The spray-dried granulate obtained is preferably a fine powder having the following particle size distribution:
d₁₀: ≦20 μm, preferably ≦10 μm
d₅₀: ≦80 μm, preferably 20 to 55 μm
d₉₀: ≦350 μm, preferably 50 to 150 μm

After spray-drying, the active ingredient telmisartan as well as the excipients contained in the spray-dried granulate are in a substantially amorphous state with no crystallinity being detectable. From a physical point of view, the spray-dried granulate is a solidified solution or glass having a glass transition temperature T_{g} of preferably >50° C., more preferably >80° C.

Based on 100 parts by weight of active ingredient telmisartan, the spray-dried granulate preferably contains 5 to 200 parts by weight of basic agent and, optionally, solubilizer and/or crystallization retarder.

The water-soluble diluent is generally employed in an amount of 30 to 95 wt. %, preferably 60 to 80 wt. %, based on the weight of the first tablet layer composition.

The lubricant is generally added to the premix in an amount of 0.1 to 5 wt. %, preferably 0.3 to 2 wt. %, based on the weight of the first tablet layer composition.

Mixing is carried out in two stages, i.e., in a first mixing step the spray-dried granulate and the diluent are admixed using, e.g., a high-shear mixer or a free-fall blender, and in a second mixing step the lubricant is blended with the premix, preferably also under conditions of high shear. The method of the invention is however not limited to these mixing procedures and, generally, alternative mixing procedures may be employed in steps c), d), and also in the subsequent steps f) and g), such as, e.g., container mixing with intermediate screening.

To provide a second tablet layer composition comprising HCTZ the constituent components may be prepared by dry-mixing, e.g., by means of a high-intensity mixer or a free-fall blender. Alternatively and preferably, the second tablet layer composition is prepared using a wet granulation technique wherein an aqueous solution of a wet granulation binder is added to a premix and subsequently the wet granulate obtained is dried, e.g., in a fluidized-bed dryer or drying chamber. The dried mixture is screened and then a lubricant is admixed, e.g., using a tumbling mixer or free-fall blender.

First and second tablet layer compositions as described above can be compressed into bilayer tablets of the target tablet weight with appropriate size and crushing strength, using an appropriate tablet press, e.g., a rotary press in the bilayer tabletting mode. Optional an appropriate external lubricant spray system for dies and punches can be used during manufacturing of tablets in order to improve lubrication. In order to avoid any cross-contamination between the tablet layers (which could lead to decomposition of HCTZ), any granulate residues should be carefully removed during tabletting by intense suction of the die table within the tabletting chamber.

In addition to the treatment of hypertension the composition according to the present invention can also be used to treat or prevent a condition selected from the group consisting of stroke, myocardial infarction, transient ischemic attack, congestive heart failure, cardiovascular disease, insulin resistance, impaired glucose tolerance, pre-diabetes, type 2 diabetes mellitus, metabolic syndrome (syndrome X), obesity, hypertriglyceridemia, elevated serum concentrations of C-reactive protein, elevated serum concentrations of lipoprotein(a), elevated serum concentration of homocysteine, elevated serum concentration of low-density lipoprotein (LDL)-cholesterol, elevated serum concentration of lipoprotein-associated phospholipase (A2), reduced serum concentration of high density lipoprotein (HDL)-cholesterol, reduced serum concentration of HDL(2b)-cholesterol, reduced serum concentration of adiponectin, cognitive decline, and dementia.

In order to further illustrate the present invention, the following non-limiting examples are given.

### EXAMPLES

### Example 1

### Surprising Responder Rate Using 80 mg Telmisartan+25 mg HCTZ

An important goal of up-titrating antihypertensive medication is to increase the number of patients adequately responding to treatment. Surprisingly improved responder rates have become obvious when the current clinical database of telmisartan was analyzed, in particular with respect to compare the responder rates upon treatment with telmisartan 80 mg/HCTZ 25 mg to those with telmisartan 80 mg/HCTZ 12.5 mg. Responders are defined as having DBP<90 mmHg or a reduction of at least 10 mmHg Regarding SBP an adequate response is defined as SBP<140 or a reduction of at least 10 mmHg When comparing telmisartan 80 mg/HCTZ 25 mg with telmisartan 80 mg/HCTZ 12.5 mg and applying the above mentioned definitions the diastolic (DBP) response rates increased by 6.6% for patients in controlled studies and by 15.4% for patients from follow-up studies. Systolic (SBP) response rates improved by 7.8% for patients from controlled and from follow-up studies.

The tables below show the detailed blood pressure response data for telmisartan 80 mg/HCTZ 25 mg in comparison to telmisartan 80 mg/HCTZ 12.5 mg from the project database:

TABLE 1
Diastolic Responder Rates
Diastolic Blood Pressure

| | | | |
|---|---|---|---|
| | | Rate of | Rate of |
| Treatment | N | Non-Responder | Responder |

Patients from Controlled Studies

| | | | |
|---|---|---|---|
| T80/H12.5 | 528 | 35.0% | 65.0% |
| T80/H25 | 134 | 28.4% | 71.6% |

Patients from Follow-up Studies

| | | | |
|---|---|---|---|
| T80/H12.5 | 799 | 41.9% | 58.1% |
| T80/H25 | 442 | 26.5% | 73.5% |

TABLE 2
Systolic Responder Rates
Systolic Blood Pressure

| | | | |
|---|---|---|---|
| | | Rate of | Rate of |
| Treatment | N | Non-Responder | Responder |

Patients from Controlled Studies

| | | | |
|---|---|---|---|
| T80/H12.5 | 528 | 24.2% | 75.8% |
| T80/H25 | 134 | 16.4% | 83.6% |

Patients from Follow-up Studies

| | | | |
|---|---|---|---|
| T80/H12.5 | 799 | 27.3% | 72.7% |
| T80/H25 | 442 | 19.5% | 80.5% |

When comparing telmisartan 80 mg/HCTZ 25 mg with telmisartan 80 mg/HCTZ 12.5 mg and applying the more recent response criteria for systolic blood pressure of SBP<140 or a reduction of at least 20 mmHg, responder rate increase by 5.7% (from 61.5% to 67.2%) for patients from controlled studies and 9.4% (from 56.2% to 65.6%) for patients from follow-up studies.

The analysis of existing evidence from the clinical database of telmisartan suggests that the telmisartan 80 mg/HCTZ 25 mg combination does consistently provide higher clinical efficacy in terms of blood pressure lowering and, especially, responder rates. Despite an expected increase in responder rates due to the increased dose of HCTZ the extent of the observed effect for the combination of telmisartan 80 mg and HCTZ 25 mg goes far beyond what was to be expected.

### Example 2

### Composition of a 680 mg Total Weight Bilayer Tablet Comprising 80 mg of Telmisartan and 25 mg of HCTZ

| | |
|---|---|
| Ingredient | mg per tablet |

| | |
|---|---|
| Telmisartan Layer: | |
| Telmisartan | 80.000 |
| Sodium hydroxide | 6.720 |
| Polyvidone K25 | 24.000 |
| Meglumine | 24.000 |
| Purified water | (400.000) |
| Sorbitol | 337.280 |
| Magnesium stearate | 8.000 |
| Total Telmisartan Layer | 480.000 |
| Hydrochlorothiazide Layer: | |
| Hydrochlorothiazide | 25.000 |
| Lactose monohydrate | 99.100 |
| Microcrystalline cellulose | 64.000 |
| Maize starch | 6.000 |
| Ferric oxide | 0.900 |
| Sodium starch glycolate | 4.000 |
| Purified water | (64.000) |
| Magnesium stearate | 1.000 |
| Total Hydrochlorothiazide Layer | 200.000 |

### Example 3

### Confirmation of the Surprisingly High Responder Rate Using 80 mg of Telmisartan+25 mg of HCTZ in a Separate Clinical Trial

To confirm the surprising responder rate of 80 mg telmisartan+25 mg HCTZ found upon analyzing the available clinical telmisartan database, the corresponding responder rate was determined for a clinical trial actually designed to compare the safety and efficacy of a 80 mg telmisartan+25 mg hydrochlorothiazide combination with the currently available 160 mg valsartan+25 mg hydrochlorothiazide combination in patients with Stage 1 and Stage 2 hypertension (confirmation study). This study was a randomized, double-blind, double-dummy, placebo-controlled, forced-titration trial, with a total duration of up to 12 weeks (eight weeks active treatment). The target population included both male and female hypertensive patients, at least 18 years of age.

The primary objective of this study was to show that the combination of MICARDIS® HCT (telmisartan 80 mg/hydrochlorothiazide 25 mg) is superior to placebo in lowering DBP and SBP, at least as effective as DIOVAN® HCT (valsartan 160 mg/hydrochlorothiazide 25 mg) in lowering DBP, and possibly superior to DIOVAN® HCT in lowering DBP and SBP in patients with Stage 1 and Stage 2 hypertension as measured by seated trough cuff blood pressure.

The primary endpoints were the change from baseline (Visit 2) for in-clinic mean seated trough cuff diastolic (DBP) and systolic blood pressure (SBP) at the end of an eight week (Visit 6) treatment period (i.e., two weeks treatment with MICARDIS® 80 mg or DIOVAN® 160 followed by six weeks treatment MICARDIS® HCT 80/25 mg or DIOVAN® HCT 160/25 mg respectively, or placebo for the entire eight weeks).

Measurements of blood pressure were made at trough (i.e., within 23-26 hours after most recent intake of the study medication).

Secondary efficacy endpoints in this study, as measured by in-clinic trough cuff blood pressure at the end of an eight week treatment period included:
1) The percentage of patients responding to treatment based on mean seated trough cuff measurements defined as:
   DBP Control: Mean seated DBP<90 mmHg at trough
   DBP Response: Mean seated DBP<90 mmHg at trough and/or a change from baseline of ≧10 mmHg
   SBP Response: Mean seated SBP<140 mmHg at trough and/or a change from baseline of ≧10 mmHg
   Normal BP: Mean seated SBP<130 mmHg at trough and mean seated DBP<85 mmHg at trough
   High Normal: Mean seated SBP≧130 and <140 mmHg at trough and a mean seated DBP≧85 and <90 mmHg at trough
2) The percentage of patients with uncontrolled HTN defined as systolic BP≧180 mmHg and/or diastolic BP≧120 mmHg at the end of study.

Safety was evaluated through the review of adverse events and through the measurement of changes from the baseline in physical examinations, laboratory parameters and vital signs (mean SBP, mean DBP) and pulse rate. At any time during the study, patients with a mean in-clinic SBP≧180 mmHg and/or DBP≧120 mmHg were to be withdrawn from the study for safety reasons. The mean value was calculated from three successive in-clinic blood pressure measurements taken two minutes apart after resting quietly in the seated position for five minutes.

Patient inclusion criteria were:

1. Ability to provide written informed consent.
2. Age 18 years or older.
3. Ability to stop current antihypertensive therapy without unacceptable risk to the patient (investigator's discretion).
4. Seated cuff DBP of ≧95 mmHg at Visit 2 (baseline).

Patient exclusion criteria were:

1. Pre-menopausal women (last menstruation≦1 year prior to start of run-in period) who:
a. were not surgically sterile; and/or
b. were nursing or pregnant; and/or
c. were of child-bearing potential and were NOT practicing acceptable means of birth control, did NOT plan to continue using this method throughout the study and did NOT agree to submit to periodic pregnancy testing during participation in studies of >three-months duration. Acceptable methods of birth control included oral, implantable or injectable contraceptives.
   2. Known or suspected secondary hypertension.
   3. Mean sitting SBP>180 mmHg or mean sitting DBP>120 mmHg at any time during the study.
   4. Hepatic and/or renal dysfunction as defined by the following laboratory parameters:
a. SGPT (ALT) or SGOT (AST)>two times the upper limit of normal range, or
b. Serum creatinine>3.0 mg/dL or creatinine clearance <0.6 ml/sec.
   5. Bilateral renal artery stenosis, renal artery stenosis in a solitary kidney, post-renal transplant or with only one kidney.
   6. Clinically relevant hypokalaemia or hyperkalaemia.
   7. Uncorrected volume depletion.
   8. Uncorrected sodium depletion.
   9. Primary aldosteronism.
   10. Hereditary fructose intolerance.
   11. Biliary obstructive disorders, cholestatis, or moderate to severe hepatic insufficiency.
   12. Patients who had previously experienced symptoms characteristic of angioedema during treatment with ACE inhibitors or angiotensin II receptor antagonists.
   13. History of drug or alcohol dependency within six months prior to start of run-in period.
   14. Chronic administration of any medications known to affect blood pressure, except medication allowed by the protocol.
   15. Any investigational drug therapy within one month of start of run-in period.
   16. Known hypersensitivity to any component of the formulation study drugs (telmisartan, valsartan or hydrochlorothiazide).
   17. Contra-indication to a placebo run-in period (e.g., stroke within the past six months, MI, cardiac surgery, PTCA or angina within the past three months prior to start of run-in period).
   18. Any other clinical condition which, in the opinion of the principal investigator, did not allow safe completion of the protocol and safe administration of telmisartan, valsartan, or hydrochlorothiazide.
   19. Night shift workers.
   20. Clinically significant ventricular tachycardia, atrial fibrillation, atrial flutter, or other clinically relevant cardiac arrhythmias as determined by the investigator.
   21. NYHA functional class CHF III-IV.
   22. Hypertrophic obstructive cardiomyopathy, aortic stenosis, or hemodynamically relevant stenosis of aortic or mitral valve.
   23. Patients whose diabetes was unstable and uncontrolled for at least the past three months as defined by a HbAlC≧10%.
   24. Concomitant use of lithium or cholestyramine or colestipol resins (potential drug interactions with hydrochlorothiazide).
   25. History of non-compliance with prescribed medication or protocol procedures.

The analysis of the data of this study reveals, that the responder rate of 80 mg telmisartan+25 mg HCTZ (T80/H25) are even higher than after the analysis of the clinical telmisartan database. Additionally, this responder rate is higher than the responder rate of 160 mg valsartan+25 mg HCTZ (Val160/H25), the difference, however, cannot be interpreted as statistically significant.

The detailed responder rate values for 80 mg telmisartan+25 mg HCTZ (T80/H25) and 160 mg valsartan+25 mg HCTZ (Val160/H25) are:

| | T80/H25 | T80/H25 | T80/H25 | Val160/H25 |
|---|---|---|---|---|
| | (confir- | (controlled | (follow-up | (confir- |
| | mation | studies of | studies of | mation |
| | study) | Example 1) | Example 1) | study) |
| DBP Response | 82.4% | 71.6% | 73.5% | 78.7% |
| SBP Response | 87.6% | 83.6% | 80.5% | 84.8% |
| New EMEA SBP | 75.2%* | 67.2% | 65.6% | 68.7%** |
| Response Criteria | | | | |

*Overall value of the responder rates for non-black (76.6%) and black patients (70.6%)
**Overall value of the responder rates for non-black (71.6%) and black patients (59.5%)

We claim:

1. A pharmaceutical composition comprising:
(a) about 80 mg of telmisartan; and
(b) about 25 mg of hydrochlorothiazide.

2. A pharmaceutical composition that can be split into halves, the pharmaceutical composition comprising:
(a) about 160 mg of telmisartan or a salt thereof; and
(b) about 50 mg of hydrochlorothiazide.

3. The pharmaceutical composition according to one of claim 1 or 2, wherein the pharmaceutical composition is a tablet or a capsule.

4. The pharmaceutical composition according to claim 3, wherein the telmisartan or a salt thereof is in a dissolving tablet matrix having instant release characteristics.

5. The pharmaceutical composition according to claim 3, wherein the hydrochlorothiazide forms a separate layer in a disintegrating pharmaceutical matrix.

6. The pharmaceutical composition according to claim 3, wherein the telmisartan or a salt thereof is in a substantially amorphous form.

7. The pharmaceutical composition according to claim 3, further comprising sodium hydroxide, meglumine, povidone, sorbitol, magnesium stearate, lactose monohydrate, microcrystalline cellulose, maize starch, and sodium starch glycolate.

8. The pharmaceutical composition according to claim 4, wherein the dissolving tablet matrix comprises a basic agent and a water-soluble diluent.

9. The pharmaceutical composition according to claim 8, further comprising other excipients and adjuvants.

10. The pharmaceutical composition according to claim 8, wherein the basic agent is selected from alkali metal hydroxides, basic amino acids, and meglumine.

11. The pharmaceutical composition according to claim 8, wherein the water-soluble diluent is selected from monosaccharides, oligosaccharides, and sugar alcohols.

12. The pharmaceutical composition according to claim 9, wherein the other excipients and adjuvants are selected from binders, carriers, fillers, lubricants, flow control agents, crystallization retarders, solubilizers, coloring agents, pH control agents, surfactants, and emulsifiers.

13. The pharmaceutical composition according to claim 8, wherein the tablet matrix is produced by spray-drying an aqueous solution comprising telmisartan and a basic agent to obtain a spray-dried granulate, mixing the spray-dried granulate, with a water-soluble diluent to obtain a premix, mixing the premix with a lubricant to obtain a final blend, and compressing the final blend to form the first tablet layer.

14. The pharmaceutical composition according to claim 5, wherein the disintegrating tablet matrix comprises a filler, a binder, and a disintegrant.

15. The pharmaceutical composition according to claim 14, further comprising other excipients and adjuvants.

16. The pharmaceutical composition according to claim 15, wherein the other excipients and adjuvants are selected from carriers, diluents, lubricants, flow control agents, solubilizers, antioxidants, coloring agents, pH control agents, surfactants, and emulsifiers.

17. The pharmaceutical composition according to claim 1, wherein the pharmaceutical composition is packaged in a moisture proof packaging material such as aluminum foil blister packs, polypropylene tubes, or HDPE bottles.

18. A method of treating hypertension in a patient in need thereof comprising administering to the patient a pharmaceutical composition comprising about 25 mg of hydrochlorothiazide and about 80 mg of telmisartan or a salt thereof.

19. A method according to claim 18, wherein the patient's blood pressure is not adequately controlled by therapy with an angiotensin II receptor antagonist or by combination of the angiotensin II receptor antagonist and a lower dose of HCTZ.

20. The method according to claim 19, wherein the patient has a low plasma renin activity or plasma renin concentration.

21. The method according to claim 18, further comprising additionally treating or preventing a condition selected from the group consisting of stroke, myocardial infarction, transient ischemic attack, congestive heart failure, cardiovascular disease, insulin resistance, impaired glucose tolerance, pre-diabetes, type 2 diabetes mellitus, metabolic syndrome (syndrome X), obesity, hypertriglyceridemia, elevated serum concentrations of C-reactive protein, elevated serum concentrations of lipoprotein(a), elevated serum concentration of homocysteine, elevated serum concentration of low-density lipoprotein (LDL)-cholesterol, elevated serum concentration of lipoprotein-associated phospholipase (A2), reduced serum concentration of high density lipoprotein (HDL)-cholesterol, reduced serum concentration of HDL(2b)-cholesterol, reduced serum concentration of adiponectin, cognitive decline and dementia.
